(19) United States
(12) Patent Application Publication
(10) Pub. No.: US 2013/0089551 A1
(43) Pub. Date: Apr. 11, 2013

(54) TREATMENT OF PULMONARY EDEMA

(71) Applicant: VEGENICS PTY LIMITED, Toorak (AU)

(72) Inventors: Marc G. Achen, North Melbourne (AU); Stephen A. Stacker, North Balwyn (AU); Teruhiko Sato, Tokyo (JP)

(73) Assignee: VEGENICS PTY LIMITED, Toorak (AU)

(21) Appl. No.: 13/646,551

(22) Filed: Oct. 5, 2012

Related U.S. Application Data
(63) Continuation of application No. 12/995,389, filed on Feb. 22, 2011, now abandoned.
(60) Provisional application No. 61/057,665, filed on May 30, 2008.

(51) Int. Cl. A61K 39/395; A61K 31/7052; A61K 38/02

(52) U.S. Cl. CPC A61K 39/39541; A61K 38/02; A61K 31/7052
USPC 424/135.1; 424/172.1; 514/1.1; 514/44 R; 424/143.1; 424/133.1; 424/136.1

(57) ABSTRACT

The invention relates to a method of treatment of pulmonary edema, comprising the step of administering to a subject in need thereof a therapeutically effective amount of an agent capable of inhibiting signaling mediated by VEGFR-3, inhibiting lymphangiogenesis or inhibiting VEGF-D activity in the subject. The method may comprise the step of identifying a subject at risk of developing pulmonary edema. The invention also relates to an agent capable of inhibiting signaling mediated by VEGFR-3, inhibiting lymphangiogenesis, or inhibiting VEGF-D activity for treating pulmonary edema in a subject. Further, the invention relates to use of an agent capable of inhibiting signaling mediated by VEGFR-3, inhibiting lymphangiogenesis, or inhibiting VEGF-D activity in the manufacture of a medicament for treating pulmonary edema in a subject.

### FIELD

The invention relates to methods of treatment of pulmonary edema.

### BACKGROUND

Edema is swelling in any organ caused by increased interstitial fluid. Increased secretion of fluid into or impaired removal of fluid from the interstitium may upset interstitial fluid homeostasis, thereby causing edema. Edema can be caused by: (1) increased hydrostatic pressure; (2) reduced oncotic pressure (osmotic pressure due to plasma proteins); (3) lymphatic obstruction; (4) destruction or removal of lymph vessels (e.g. by radiotherapy or surgery (5) sodium retention; or (6) inflammation (e.g. from infection). Loss of lymphatic function causes lymphedema.

The circulatory system by which blood travels comprises the heart, arteries, arterioles, and capillaries. Capillary networks promote gas and metabolite exchange, after which blood is returned via venules and veins to the heart and lungs where it is replenished with oxygen.

The lymphatic vasculature transports fluid and macromolecules from tissues back to the blood circulation. It forms a unidirectional network that collects interstitial fluid to be returned to the venous circulation via collecting vessels, lymph nodes, lymphatic trunks and ducts. The lymphatic vasculature also links tissue fluids to lymph nodes as an immune surveillance system by which lymphocytes and antigen-presenting dendritic cells travel from peripheral tissues to the lymphoid organs, in which immune responses against pathogens are launched. The formation of lymphatic vessels, particularly from pre-existing lymphatic vessels, is known as lymphangiogenesis.

The endothelial cells of blood and lymphatic vessels share numerous elements. However, a continuous basement membrane is absent from the endothelium of lymphatic capillaries, and the intercellular junctions are not sealed tightly. Interstitial fluid accumulation opens intercellular junctions to promote interstitial fluid uptake by the lymph vessel.

Starling's equation describes the generation of interstitial fluid by the competition of hydrostatic and oncotic forces across semipermeable capillary walls. Thus, increased hydrostatic pressure or reduced oncotic pressure within a blood vessel, or increased capillary permeability, will tend to promote interstitial fluid volume and consequently edema.

Pulmonary edema is a subclass of edema involving swelling of the lungs or accumulation of fluid in the lungs. Pulmonary edema is caused by impaired removal of fluid from the lungs by the heart, or by lung injury per se (direct or indirect), and thus can be categorized as either cardiogenic or non-cardiogenic, respectively. Pulmonary edema causes a severe drop in the oxygen content of the blood due to fluid in the lung acting as a diffusion barrier between gas in the lungs and the blood with which that gas normally equilibrates. Pulmonary edema can often be fatal within a matter of minutes.

Conflicting data and assertions have been published on the role of VEGF-C and VEGF-D in pulmonary function and pathological conditions associated with incorrect pulmonary function. For example, and on the one hand, it has been suggested that a peptide inhibitor of VEGF-D or VEGF-C, or an inhibitor or antibody antagonist of VEGF-D, may be used in a method of treating fluid accumulation in the lungs (WO2001/52875, U.S. Pat. No. 6,235,713 B1). In contrast, and more recently, it has been suggested that inhibiting VEGFR-3 signaling may interfere with fluid clearance and result in, for example, exaggerated mucosal edema (e.g. bronchial lymphedema) (Baluk et al., 2005, J. Clin. Invest., 115(2), 247-257). The inventors have now found that the promotion of vascular permeability would appear not to be the primary role of VEGF-C and VEGF-D, and in particular VEGF-D, in the lung.

Despite the urgent need to efficiently treat pulmonary edema, there is no efficient therapeutic agent available for treating pulmonary edema. Treatment for pulmonary edema generally involves maximizing respiratory function and depends upon removal of the underlying cause of the pulmonary edema. Therefore, a need exists for an effective, alternative means to prevent, restrict or treat pulmonary edema in clinical settings.

### SUMMARY

According to a first aspect, the invention provides a method of treatment of pulmonary edema, comprising the step of administering to a subject in need thereof a therapeutically effective amount of an agent capable of inhibiting signaling mediated by vascular endothelial growth factor receptor 3 (VEGFR-3) in the subject.

According to a second aspect, the invention provides a method of treatment of pulmonary edema, comprising the step of administering to a subject in need thereof a therapeutically effective amount of an agent capable of inhibiting lymphangiogenesis in the subject.

According to a third aspect, the invention provides a method of treatment of pulmonary edema, comprising the step of administering to a subject in need thereof a therapeutically effective amount of an agent capable of inhibiting VEGF-D activity in the subject.

The inventors have discovered that genetic insufficiency of Vegfd in a mouse model, caused by inactivation of the Vegfd gene, protected the so called Vegfd “knockout” mice from pulmonary edema compared with wild-type control mice, when each group of mice was challenged to hyperoxic conditions.

Vegfd and its encoded protein product VEGF-D are central to signaling mediated by VEGFR-3, and to lymphangiogenesis. Signaling mediated by VEGFR-3 and lymphangiogenesis are believed to be important for removal of interstitial fluid. Therefore, it is unexpected and surprising that inhibition of signaling mediated by VEGFR-3, or lymphangiogenesis, is beneficial for treating pulmonary edema.

According to a fourth aspect, the invention provides a method of treatment of pulmonary edema, comprising the steps of:
identifying a subject at risk of developing pulmonary edema; and
administering to the subject a therapeutically effective amount of an agent capable of inhibiting signaling mediated by VEGFR-3 in the subject.

According to a fifth aspect, the invention provides a method of treatment of pulmonary edema, comprising the steps of:
identifying a subject at risk of developing pulmonary edema; and
administering to the subject a therapeutically effective amount of an agent capable of inhibiting lymphangiogenesis in the subject.

According to a sixth aspect, the invention provides a method of treatment of pulmonary edema, comprising the steps of:
identifying a subject at risk of developing pulmonary edema; and
administering to the subject a therapeutically effective amount of an agent capable of inhibiting VEGF-D activity in the subject.

The inventors contemplate exercising the invention to treat pulmonary edema. However, there are many conditions of both cardiogenic and non-cardiogenic etiology that give rise to pulmonary edema. Such conditions can be classified also according to whether or not they require emergency treatment.

In one sense, pulmonary edema can be considered a symptom, side-effect or complication of such conditions. Many of the conditions exhibit acute onset and require emergency clinical intervention. Emergency onset of the condition is a trigger for development of pulmonary edema, which in itself is a life-threatening indication. Thus, acute or emergency onset of the critical event is a “risk” for developing pulmonary edema. However, chronic existence of a condition associated with development of pulmonary edema is also a “risk” for developing pulmonary edema.

Because such risks can trigger development of pulmonary edema, the inventors envisage practice of the invention as an adjunct therapy or co-therapy when treatment of the causative condition by clinical intervention begins. Therefore, it is envisaged that an agent that reduces VEGF-D activity, reduces signaling mediated by VEGFR-3 or that reduces lymphangiogenesis will be administered before, in the case of elective clinical intervention, or as soon as is medically practicable after the risk of developing pulmonary edema is identified, in the case of emergency clinical intervention.

A seventh aspect provides an agent capable of inhibiting signaling mediated by VEGFR-3, inhibiting lymphangiogenesis, or inhibiting VEGF-D activity for treating pulmonary edema in a subject.

An eighth aspect provides use of an agent capable of inhibiting signaling mediated by VEGFR-3, inhibiting lymphangiogenesis, or inhibiting VEGF-D activity in the manufacture of a medicament for treating pulmonary edema in a subject.

In a preferred embodiment according to any aspect of the invention, the agent is administered to the subject as soon as is medically or clinically practicable after pulmonary edema initiates. Alternatively, the agent is administered to the subject as soon as is medically or clinically practicable after the risk of developing pulmonary edema is identified.

In a particularly preferred embodiment according to the fourth, fifth or sixth aspect of the invention, the agent is administered to the subject before pulmonary edema initiates.

In a preferred embodiment according to any aspect of the invention, the agent is an inhibitor of signaling mediated by VEGFR-3. Alternatively, the inhibitor is an inhibitor of VEGF-D activity or lymphangiogenesis.

Whilst not wishing to be bound by any particular theory, the inventors propose that reduction of signaling mediated by VEGFR-3 is central to treating pulmonary edema. Alternatively, the inventors propose that reduction of VEGF-D activity or lymphangiogenesis is central to treating pulmonary edema.

### BRIEF DESCRIPTION OF THE FIGURES

FIG. 1. Macroscopic appearance of mouse lungs after 60 hours of hyperoxia followed by a 24-hour normoxic recovery period. FIG. 1a (left) shows the lung from a wild-type control mouse. The presence of pulmonary edema is indicated by the inconsistent coloration of the tissue (arrows). FIG. 1b (right) shows the lungs from a Vegfd knockout mouse which are healthy in appearance, as indicated by the consistent coloration.

FIG. 2. Schemes outlining strategy for the hyperoxia challenge experiment in wild-type mice. “N” denotes normoxic exposure and arrows denote monoclonal antibody (mAb) injections.

### DETAILED DESCRIPTION

Edema

“Edema” refers to swelling in any organ caused by increased interstitial fluid.

“Interstitial fluid” is used interchangeably with “lymph” and refers to fluids and macromolecules, such as proteins, extravasated from blood vessels, and cells, commonly white blood cells, that collect in lymph vessels for return to the blood circulation. The lymph, or interstitial fluid, is then transported within the lymphatic vasculature.

“Lymphedema” refers to an abnormality of lymph flow and transport, usually caused by obstruction of the lymphatic system, destruction or removal of lymph vessels (e.g. by radiotherapy or surgery), or infiltration of the lymphatic system by infection. As referred to herein, lymphedema includes edema caused by increased capillary permeability, heart failure or venous insufficiency.

Pulmonary Edema

“Pulmonary edema” is a subclass of edema involving swelling of the lungs or accumulation of fluid in the lungs.

The state in which the blood possesses reduced oxygen content is known as “hypoxemia”, whereas the condition in which tissues possess reduced oxygen content is known as “hypoxia”, but as used herein, the terms may be used interchangeably.

“Emergency” or “acute” refers to an imminent threat to the health of a subject that, in the absence of immediate clinical intervention, will be detrimental to the health of the subject, will result in injury to the subject, or may even cause death of the subject. Often, an emergency arises from a triggering event caused by an underlying chronic condition.

“Elective”, “chronic” or “non-emergency” refers to a threat to the health of a subject that does not require immediate clinical intervention. Without clinical intervention, the threat will not be immediately detrimental to the health of the subject. However, trigger events related to such conditions may precipitate an emergency or acute condition from an elective, chronic or non-emergency condition.

Chronic conditions that are associated with development of pulmonary edema are to a significant degree predictable. In such cases, the risk associated with development of pulmonary edema and requiring clinical intervention may be classed as elective, or non-emergency. Because of such predictability, it will be facile in many instances to administer an agent that reduces VEGF-D activity, reduces signaling mediated by VEGFR-3, or reduces lymphangiogenesis, before pulmonary edema initiates, and before or at the same time as any clinical intervention for treating the chronic condition begins.

In contrast, by the very nature of an acute or emergency condition, it may not be possible to administer an agent before the risk or trigger eventuates. Pulmonary edema develops in response to a given condition, and treatment for pulmonary edema involves maximizing respiratory function and depends upon removal of the underlying cause. Therefore, in emergency situations, the imminent threat posed by the underlying cause or trigger event, i.e. the risk per se, may take immediate precedence so that an agent for treating pulmonary edema may not be administered within a specific time period. Ideally, administration of such an agent will occur as soon as is practicable under the conditions of a given medical emergency. Furthermore, given the myriad conditions that are associated with development of pulmonary edema, the precise medical factors will equally be many and varied. It is for these reasons that the agent is administered to the subject at about the time the risk of developing pulmonary edema is identified, where “at about” refers to as soon as practicable under any given medical environment.

As used herein, “risk” refers to the existence of a condition that is associated with or pre-disposes a subject to development of pulmonary edema. A risk may constitute a chronic or acute condition, or an acute or trigger event arising from an underlying chronic condition, any of which is associated with development of pulmonary edema. Thus, acute or emergency onset of a critical event is a “risk” for developing pulmonary edema. Similarly, existence of an elective, chronic or non-emergency condition is also a “risk” for developing pulmonary edema.

Preferably, the agent to be administered according to the invention should only be present in the subject from the time of administration to the medically determined point of maximum benefit. In practice, the pharmacokinetics of the agent will allow calculation of the optimal point of cessation of treatment with respect to maximum benefit. Alternatively, an antagonist of the therapeutic agent may be administered. In one embodiment, the cessation of treatment is facilitated by administration of VEGF-D or VEGF-C to neutralize the effect of any residual agent in the subject's system.

“Treating” or “treatment” refers to both therapeutic treatment and prophylactic or preventative measures, wherein the aim is to prevent, ameliorate or slow down (lessen) pulmonary edema.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

The “subject” includes a mammal. The mammal may be a human, or may be a domestic, zoo, or companion animal. While it is particularly contemplated that the methods of the invention are suitable for medical treatment of humans, they are also applicable to veterinary treatment, including treatment of companion animals such as dogs and cats, and domestic animals such as horses, cattle and sheep, or zoo animals such as felids, canids, bovids, and ungulates.

The term “therapeutically effective amount” refers to an amount of the agent capable of reducing VEGF-D activity, reducing signaling mediated by VEGFR-3 or reducing lymphangiogenesis in a subject or mammal to a level which is beneficial to treat pulmonary edema. A therapeutically effective amount may be determined empirically and in a routine manner, in relation to treating pulmonary edema, and will result in a reduction of lung fluid.

Cardiogenic Pulmonary Edema

In one embodiment, the invention relates to a method of treating cardiogenic pulmonary edema by administering an inhibitor of VEGF-D activity, signaling mediated by VEGFR-3 or lymphangiogenesis.

As used herein, “cardiogenic” pulmonary edema refers to the impaired removal of fluid from the lungs by the heart. Cardiogenic pulmonary edema is seen in multiple conditions including, but not limited to: arrhythmias; atherosclerosis; arteriosclerosis; arteriovenous malformation; cardiomyopathy; cardiovascular disease; congestive heart failure; coronary artery disease; hypervolemia; heart attack; left ventricular failure; heart disease; hypertensive crisis; pulmonary hypertension; pericardial effusion; pericardial tamponade; or valvular heart disease.

As used herein, “arrhythmia” refers to abnormal electrical activity in the heart so that the heart beats too fast (tachycardia), too slow (bradycardia), and may beat regularly or irregularly.

“Atherosclerosis” refers to blockage of an artery, largely comprising cholesterol, macrophages and remnants of previous blockages.

“Arteriosclerosis” refers to hardening of an artery.

“Arteriovenous malformation” (AVM) refers to an abnormal connection between arteries and veins, to some extent bypassing capillary beds, and is prone to bleeding.

“Cardiomyopathy” refers to deterioration of the heart muscle by any cause and includes “hypertrophic cardiomyopathy”. Mutations in genes encoding sarcomeric proteins may be associated with hypertrophic cardiomyopathy.

“Cardiovascular disease” refers to any disease that involves the heart or blood vessels, but can be used interchangeably with “atherosclerosis”.

“Congestive heart failure” (CHF), “congestive cardiac failure” (CCF) or “heart failure” can be used interchangeably and each refers to any structural or functional disorder of the heart that impairs its ability to fill with or pump sufficient blood through the body for the requirements of metabolizing tissues.

“Coronary artery disease” (CAD) can be used interchangeably with the terms “coronary heart disease” (CHD) or “ischemic heart disease” and each refers to an atherosclerotic blockage of the artery supplying blood to the heart muscle.

“Hypervolemia” or “fluid overload” may be caused, for example, by kidney failure or intravenous therapy, and refers to too much fluid in the blood.

“Heart attack”, “acute myocardial infarction” (AMI) or “myocardial infarction” (MI) refers to interruption of the blood supply to the heart causing oxygen shortage, or ischemia. Heart attack generally results from atherosclerosis of the coronary arteries. Heart attack causes both a change in hemodynamic effects and an alteration in structure in the damaged and healthy zones of the heart. Thus, for example, heart attack reduces the maximum cardiac output and the stroke volume of the heart.

“Left ventricular failure” refers to a failure of the left ventricle, the lower left quadrant of the heart, to pump sufficient blood into the aorta, from which the coronary arteries stem.

“Heart disease” refers generally to any disease of the heart.

“Hypertensive crisis” or “hypertensive emergency” refers to severe hypertension accompanied by acute organ impairment and the risk of irreparable organ damage.

“Pulmonary hypertension” refers to increased blood pressure in the pulmonary artery, pulmonary vein, or pulmonary capillaries, together forming the lung vasculature, and can cause right heart failure. The most common cause of pulmonary hypertension is left heart failure.

“Pericardial effusion” refers to abnormal accumulation of fluid in the space between the heart and the “pericardium” which is a sac surrounding the heart. “Cardiac tamponade” or “pericardial tamponade” refers to the situation where a pericardial effusion is great enough to affect heart function.

“Valvular heart disease”, “valvular disease”, or “valvular regurgitation” refers to disease of any of the valves of the heart. Valvular regurgitation occurs as a result of disorders of the cardiac valves. Various diseases, like rheumatic fever, can cause shrinking or pulling apart of the valve orifice, while other diseases may result in endocarditis, an inflammation of the endocardium or lining membrane of the atrioventricular orifices and adversely affect operation of the heart. Defective narrowing of the valve stenosis or defective closing of the valve results in an accumulation of blood in the heart cavity or regurgitation of blood past the valve. If uncorrected, prolonged valvular stenosis or insufficiency may result in cardiac hypertrophy and associated damage to the heart muscle. A specific case is “mitral valve disease”, “mitral regurgitation” (MR), or “mitral insufficiency”, which refers to abnormal leakage of blood through the mitral valve, from the left ventricle into the left atrium of the heart.

Non-Cardiogenic Pulmonary Edema

In another preferred embodiment, the invention relates to a method of treating non-cardiogenic pulmonary edema by administering an inhibitor of VEGF-D activity, signaling mediated by VEGFR-3 or lymphangiogenesis.

As used herein, “non-cardiogenic” pulmonary edema refers to the impaired removal of fluid from the lungs caused by direct or indirect lung injury. Non-cardiogenic pulmonary edema is seen in acute lung injury (ALI) and acute respiratory distress syndrome (ARDS), which are inflammatory disorders of the lung most commonly caused by sepsis. Non-cardiogenic pulmonary edema is also observed in, but not limited to: pneumonia; pancreatitis; trauma; aspiration; burns; inhalation of toxic or noxious gas or fumes; air embolism; amniotic fluid embolism; fat embolism; massive or multiple blood transfusions; eclampsia; poisoning; radiation; asthma; bronchopulmonary dysplasia; chronic obstructive pulmonary disease or chronic obstructive airway disease; high altitude pulmonary edema; hyperoxic acute lung injury or oxygen toxicity; hypoventilation or respiratory depression; some medications; narcotic (opioid) overdose; re-expansion pulmonary edema; reperfusion or reperfusion injury; tuberculosis; or upper airway obstruction or airway obstruction.

As used herein, “acute lung injury” (ALI) is a collective term that refers to hypoxemic respiratory failure, a severe version of which is “acute respiratory distress syndrome” (ARDS). ALI/ARDS is caused by any stimulus of local or systemic inflammation, principally sepsis. It is characterized by hypoxemia, diffuse infiltrates on chest X-ray in the absence of elevated left atrial pressure, pulmonary edema, low lung compliance and widespread capillary leakage. Artificial ventilation with a high oxygen concentration is essential to maintain adequate blood oxygenation when treating ALI/ARDS. Although the technologies for mechanical ventilation and treatment of sepsis have improved over the past ten years, mortality in ALI/ARDS remains at approximately 40%.

Lung transplantation can be successful in the treatment of patients with end-stage pulmonary disease. However, pulmonary edema following lung reperfusion during transplantation is a major clinical problem, which often results in lung transplant rejection. Thus, pneumonectomy in preparation for lung transplantation also causes a form of lung injury that can be classified as non-cardiogenic. A donor lung to undergo pneumonectomy can be considered a special case, merging elements of emergency and non-emergency conditions. Although the lung must be removed with diligence upon identification of the donor, there will be opportunity to administer an agent according to the invention to the donor or the donor lung per se. Preferably, the recipient will also be administered an agent according to the invention. Thus, in a preferred embodiment relating to lung transplantation, the donor and/or the donor lung and/or the recipient of the donor lung will be treated with an agent to reduce pulmonary edema according to the invention.

Primary ALI is caused by a direct injury to the lung (e.g. pneumonia). Unfortunately, exposure to high oxygen concentrations 50%) for prolonged periods causes hyperoxic acute lung injury (HALI), also a form of direct lung injury. HALI is characterized by endothelial/epithelial injury and pulmonary edema, which further contributes to the difficulty of treating ALI/ARDS. Similarly, supplemental oxygen is commonly administered to patients with significant pulmonary or cardiac disease to increase the delivery of oxygen to peripheral tissues, thereby potentially causing direct lung injury in those subjects also.

Secondary ALI is caused by an indirect insult (e.g. pancreatitis). Thus, additional causes of ALI/ARDS include trauma (e.g. pulmonary contusion, multitrauma, neurogenic trauma such as subarachnoid hemorrhage), aspiration (e.g. gastric fluid, drowning), burns, inhalation of toxic or noxious gas or fumes, air embolism, amniotic fluid embolism, fat embolism, massive or multiple blood transfusions, eclampsia, poisoning, or radiation.

“Sepsis” refers to the state of whole-body inflammation caused by infection.

“Compliance” refers to the ability of the lung to distend in response to pressure without disruption of structure or function.

“Pneumonia” refers to an inflammatory disease of the lung, including abnormal alveolar filling with fluid, where “alveoli” are air-filled sacs of the lungs from which oxygen is absorbed.

“Pancreatitis” refers to inflammation of the pancreas.

“Subarachnoid hemorrhage” refers to a type of bleeding into a specific space surrounding the brain.

“Embolism” refers to blockage of a blood vessel that arises from an event remote from the actual site of blockage.

“Eclampsia” refers to a convulsive state, usually during pregnancy.

“Asthma” refers to a chronic condition of the lung in which the airways constrict, become inflamed, and are lined with excessive mucus. Asthma is characterized by recurrent episodes of airway obstruction.

“Bronchopulmonary dysplasia” arises in infancy as the result of ventilator and oxygen therapy for respiratory distress syndrome after premature birth. Ventilator-induced lung injury, oxygen toxicity and inflammation are important pathogenic factors.

“Chronic obstructive pulmonary disease” (COPD) or “chronic obstructive airway disease” (COAD) refers to a pathological limitation of airflow in the airway that is not fully reversible, where the obstruction of airflow results in air becoming trapped in the lungs. COPD includes chronic bronchitis, emphysema and a range of other lung disorders.

“High altitude pulmonary edema” (HAPE) refers to a condition arising from a shortage of oxygen, which is caused by reduced pressure at high altitudes.

“Hyperoxic acute lung injury” (HALI) or “oxygen toxicity” refers to severe increases in the oxygen content of blood or tissues caused by breathing oxygen at elevated partial pressures, as during treatment for ALI/ARDS, for example, and wherein the oxygen damages cells.

“Hypoventilation” or “respiratory depression” refers to inadequate ventilation to maintain gas exchange in the lung. Hypoventilation can be caused by a range of central nervous system depressant drugs, but particularly after narcotic (opioid) overdose.

“Re-expansion pulmonary edema” refers to too rapid expansion of the lung that may occur after large volume thoracentesis or pneumonectomy. “Thoracentesis” refers to an invasive procedure to remove fluid (pleural effusion) or air (pneumothorax or collapsed lung) from the chest cavity outside the lung. “Pneumonectomy” refers to surgical removal of the lung.

“Reperfusion” or “reperfusion injury” refers to tissue damage caused by restoration of the blood supply after a period of ischemia, or absence of oxygen. The absence of oxygen and nutrients from blood creates a condition in which rather than normal function, the restored blood supply causes inflammation and oxidative damage. This can occur after pulmonary thromboendartectomy to remove a blockage from any of the pulmonary arteries to restore blood flow to the lungs, or can occur after lung transplantation.

“Tuberculosis” is a common and deadly infectious disease caused by mycobacteria, mainly Mycobacterium tuberculosis.

“Upper airway obstruction” or “airway obstruction” refers to increased resistance in the lung that reduces the inhaled volume of oxygen and thereby reduces the amount of oxygen absorbed during breathing.

Lymphangiogenesis

“Lymphangiogenesis” refers to formation of lymphatic vessels, particularly from pre-existing lymphatic vessels, but as used herein, the term applies to formation of lymph vessels under any condition. It also applies to the enlargement of lymphatic vessels, commonly known as lymphatic hyperplasia.

Lymphangiogenesis is regulated to a large extent by VEGF-C and VEGF-D. Lymphangiogenesis appears to be regulated by signaling mediated by VEGFR-3, particularly upon specifically binding its ligands, VEGF-C and VEGF-D. VEGF-C and VEGF-D are two of six members of a family of angiogenic regulators. Other members are VEGF-A (also known as VEGF), VEGF-B, VEGF-E and placental growth factor (PlGF).

During embryogenesis, lymphatic endothelial cell sprouting, proliferation and survival is promoted by VEGF-C. Lymphatic vessels fail to develop in mice in which VEGF-C is absent (Vegfc knockout mice), and such mice develop severe edema. Indeed, absence of VEGF-C is embryonic lethal. Lymphatic vessel hypoplasia and lymphedema is exhibited in the skin of mice hemizygous for Vegfc (i.e. mice possessing one functional allele).

Lymphangiogenesis is also partly regulated by VEGF-D, similar to VEGF-C. However, lymphangiogenesis during embryonic development is not dependent upon VEGF-D, as demonstrated by Vegfd knockout mice. The lymphatic system in Vegfd knockout mice is relatively normal and Vegfd knockout mice are viable and fertile. The absolute abundance of lymphatic vessels in the lung is, however, reduced by approximately 30% compared to wild-type mice.

Lymphatic vessels express VEGFR-3, the receptor for VEGF-C and VEGF-D, and both VEGF-C and VEGF-D signal predominantly through VEGFR-3. VEGF-C and VEGF-D are proteolytically processed by proprotein convertases. In humans, mature proteolytically processed forms of VEGF-C and VEGF-D also bind to VEGFR-2. In mice, VEGF-D binding is restricted to VEGFR-3.

VEGF-C and VEGF-D exist as homodimers, and it has been suggested that they may exist as VEGF-C-VEGF-D heterodimers. In addition to lymphatic vessels, VEGFR-3 is also expressed on blood vessel endothelial cells during development, thereby accounting for the severe vasculogenic and angiogenic defects observed during early embryogenesis in models comprising inactive VEGFR-3 signaling. The lymphatic system possesses almost exclusive expression of VEGFR-3 in adulthood, because VEGFR-3 expression in blood vessels declines following birth and during adolescence. Thus, only lymphangiogenesis is inhibited in adults by inhibition of the VEGF-C-VEGF-D-VEGFR-3 signaling axis.

Lymphatic vessels express neuropilin-2 (NRP-2), which can bind VEGF-C or VEGF-D. In lymphangiogenesis, NRP-2 is thought to act as a co-receptor to increase the binding affinity of VEGF-C or VEGF-D to VEGFR-3. NRP-2 is required for lymphangiogenesis. Proliferation of lymphatic vessel endothelial cells was reduced and lymphatic vessels and capillaries failed to develop in Nrp2 knockout mice in which NRP-2 is absent. Similarly, NRP-1 is capable of binding VEGF-C and VEGF-D.

Defective lymphatic capillaries are the underlying cause of Milroy disease and other rare hereditary forms of lymphedema in humans. Tyrosine kinase-inactivating point mutations of the VEGFR-3 gene have been identified as a major cause of Milroy disease, and VEGF-C and VEGF-D therapy has shown promising efficacy in preclinical animal models. However, previous work has only demonstrated lymphatic capillary reconstitution, whereas effects on the collecting lymphatic vessels that are more commonly damaged in lymphedema have not been addressed.

Lymphatic vessel growth in adult tissues can be induced by Angiopoietin-1 (ANG-1) through its binding to the tunica interna endothelial cell kinase receptor 2 (TIE-2 or TEK). Lymphatic vessel sprouting that was induced by ANG-1 was inhibited by an inhibitor of VEGFR-3. Furthermore, VEGFR-3 was up-regulated by ANG-1 binding to TIE-2. TIE-2 expressed on lymphatic vascular endothelial cells may also be agonized by ANG-2 and ANG-3.

VEGF-C and VEGF-D may act as ligands for integrins. Specifically, VEGF-C and VEGF-D have been shown to act as ligands for integrin α9β1. Cell adherence and cell migration were promoted by each of VEGF-C and VEGF-D in cells expressing integrin α9β1. The effect could be blocked by an anti-integrin α9β1 antibody or siRNA directed to integrin α9β1.

Thus, in lymphangiogenesis, VEGFR-3 appears to be central. VEGFR-3 specifically binds and is activated by ligands VEGF-C and VEGF-D. VEGF-C and VEGF-D are synthesized as prepro-polypeptides and are activated by proteolytic processing by proprotein convertases. VEGF-C and VEGF-D also bind specifically to NRP-2, which is though to be a co-receptor for VEGFR-3. Both lymphangiogenesis and VEGFR-3 are up-regulated when ANG-1 specifically binds to TIE-2. It is thought that binding of VEGF-C or VEGF-D to integrins, particularly integrin α9β1, also performs a role in lymphangiogenesis.

Despite the implication that VEGFR-3 is central to lymphangiogenesis, the inventors are not bound to the particular theory that reduction of lymphangiogenesis is the only mechanism for treatment of pulmonary edema. The inventors also contemplate that reduction of signaling mediated by VEGFR-3 per se may also be a mechanism for treatment of pulmonary edema. Furthermore, the inventors contemplate that reduction in VEGF-D activity per se may be a treatment for pulmonary edema.

Inhibitors

Persons skilled in the art will appreciate from the foregoing that inhibition of VEGF-D activity, inhibition of signaling mediated by VEGFR-3 or inhibition of lymphangiogenesis can occur at a variety of biological points comprising any one or more of the interactions described.

An “inhibitor” is any substance that partially or fully blocks, neutralizes, reduces or antagonizes a biological activity of a molecular component of signaling mediated by VEGFR-3 or lymphangiogenesis. Alternatively, an inhibitor is any substance that partially or fully blocks, neutralizes, reduces or antagonizes a VEGF-D biological activity. Thus, “inhibition” is the corresponding state elicited by an inhibitor. A molecular component of signaling mediated by VEGFR-3 or lymphangiogenesis includes VEGFR-3, VEGF-C, VEGF-D, proprotein convertases, neuropilin-1 (NRP-1), neuropilin-2 (NRP-2), angiopoietin-1 (ANG-1), tunica interna endothelial cell kinase receptor (TIE-2) or integrin α9β1.

It is envisaged that practice of the invention extends to any inhibitor known now or in the future.

Suitable classes of inhibitor molecules that target VEGF-D or signaling mediated by VEGFR-3, or lymphangiogenesis include antibodies, polypeptides, peptides, peptide mimetics, nucleic acid molecules, and small molecules. Such classes of inhibitor molecules are suitable also for inhibiting binding of ligands, for example VEGF-C or VEGF-D, to integrins, particularly integrin α9α1.

Suitable VEGF-D, VEGFR-3-mediated signaling or lymphangiogenesis antibody inhibitors include antagonist and neutralizing antibodies or antibody fragments.

Polypeptide, peptide, or peptide mimetic VEGF-D inhibitors, VEGFR-3-mediated signaling inhibitors or lymphangiogenesis inhibitors include fragments or amino acid sequence variants of native polypeptide or peptide components of VEGF-D, VEGFR-3-mediated signaling or lymphangiogenesis.

Nucleic acid molecule inhibitors of VEGF-D activity, VEGFR-3-mediated signaling or lymphangiogenesis include antisense molecules, nucleic acids in triple-helix formation, small interfering RNA (siRNA), and ribozymes.

Small molecule inhibitors of VEGF-D, VEGFR-3-mediated signaling or lymphangiogenesis include organic and inorganic molecules.

Inhibitors of VEGF-D activity, VEGFR-3-mediated signaling or lymphangiogenesis according to the present invention may exert their effects by interacting with any one or more of VEGFR-3, VEGF-C, VEGF-D, proprotein convertases, NRP-1, NRP-2, ANG-1, TIE-2 or integrins, particularly integrin α9α1, in their DNA, RNA or polypeptide forms.

Inhibition of VEGF-D activity, VEGFR-3-mediated signaling or lymphangiogenesis according to the present invention may occur via inhibition of ligand availability for receptor binding, inhibition of receptor availability for ligand binding, inhibition of receptor tyrosine kinase activity, or inhibition of co-receptor interaction.

As used herein, “availability” refers to the potential or actual amount of a molecule that performs some function in VEGF-D activity, VEGFR-3-mediated signaling or lymphangiogenesis and is present in a biological system. Availability may be relative or absolute. For example, if all copies of a gene encoding a polypeptide involved in lymphangiogenesis were rendered non-functional by genetic mutation and no functioning polypeptide was synthesized, then there would be no availability of the polypeptide in an absolute sense. Alternatively, if the same gene was present with one functioning copy and 50% of the polypeptide was synthesized, there would be reduced or inhibited availability in a relative sense. Similarly, other mechanisms may be envisaged where availability is affected. Receptors may be transcribed or translated to a lesser degree when compared with a control, or the receptor may be targeted by an antibody that binds specifically to the ligand binding site, thereby reducing or inhibiting receptor availability for ligand binding. Analogously, if ligand synthesis is targeted by an antisense inhibitor, or if an antibody inhibitor or soluble receptor inhibitor specifically binds to the ligand, then there will be reduction or inhibition of ligand availability for receptor binding.

The term “specific binding” or “specifically binds” or “specific for” refers to binding where a molecule binds to a particular polypeptide or epitope on a particular polypeptide without substantially binding to any other polypeptide or polypeptide epitope. Such binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. As used herein, specific binding is used in relation to the interaction between the molecular components of VEGF-D activity, VEGFR-3-mediated signaling or lymphangiogenesis. Specific binding is also used in relation to the interaction between the molecular components of VEGF-D activity, VEGFR-3-mediated signaling or lymphangiogenesis and agents that partially or fully block, neutralize, reduce or antagonize a biological activity of a molecule that facilitates VEGFR-3-mediated signaling or lymphangiogenesis. Specific binding also applies to the interaction between the molecular components of VEGF-D activity and agents that partially or fully block, neutralize, reduce or antagonize VEGF-D biological activity.

In particular, specific binding refers to a molecule having a K_{d} at least 2-fold less for the particular polypeptide or epitope on a particular polypeptide than it does for a non-specific target. Preferably, specific binding refers to a molecule having a Kd at least 4-fold, 6-fold, 8-fold or 10-foldless for the particular polypeptide or epitope on a particular polypeptide than it does for a non-specific target. Alternatively, specific binding can be expressed as a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹° M, alternatively at least about 10⁻¹¹ M, alternatively at least about 10⁻¹² M, or less.

The person skilled in the art will appreciate that there exist many mechanisms for inhibiting VEGF-D activity, VEGFR-3-mediated signaling or lymphangiogenesis. The principal aim is to reduce receptor signaling. Some examples will be described below, but such a list is not intended to be limiting.

Antibody Inhibitors

The term “antibody” is used in the broadest sense and specifically covers, for example, polyclonal antibodies, monoclonal antibodies (including antagonist and neutralizing antibodies), antibody compositions with polyepitopic specificity, single chain antibodies, and fragments of antibodies, provided that they exhibit the desired biological or immunological activity.

An “antibody inhibitor” will specifically bind to a particular polypeptide or epitope on a particular polypeptide without substantially binding to any other polypeptide or polypeptide epitope. Such binding will partially or fully block, neutralize, reduce or antagonize VEGF-D activity or a biological activity of a molecule that facilitates VEGFR-3-mediated signaling or lymphangiogenesis. Such target molecules include VEGFR-3, VEGF-C and VEGF-D, for example.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. Generally, the antibody will be purified (1) to greater than 95% by weight of antibody as determined by the Lowry method, and most preferably more than 99% by weight, (2) to a degree sufficient to obtain at least 15 residues of N terminal or internal amino acid sequence by use of a spinning cup sequenator, or (3) to homogeneity by SDS PAGE under reducing or non-reducing conditions using Coomassie blue or, preferably, silver stain. An isolated antibody includes the antibody in situ within recombinant cells since at least one component of the antibody's natural environment will not be present. Ordinarily, however, isolated antibody will be prepared by at least one purification step.

Where antibody fragments are used, the smallest inhibitory fragment that specifically binds to the binding domain of the target protein is preferred.

Polyclonal Antibodies

Polyclonal antibodies are preferably raised in animals by multiple subcutaneous (s.c.) or intraperitoneal (i.p.) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

In one protocol for generating polyclonal antibodies, animals are immunized against the antigen, immunogenic conjugate, or derivative, by combining the antigen, conjugate or derivative with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method.

Monoclonal antibodies may be made using the hybridoma method in which a mouse or other appropriate host animal, such as a hamster, is immunized to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium, which preferably contains one or more substances that inhibit the growth or survival of the unfused, parental myeloma cells (also referred to as fusion partner).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

Once hybridoma cells that produce antibodies of the desired specificity, affinity, and/or activity are identified, the clones may be subcloned by limiting dilution procedures and grown by standard methods. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxyapatite chromatography, gel electrophoresis, or dialysis.

DNA encoding the monoclonal antibodies is readily isolated and sequenced using conventional procedures. The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

Monoclonal antibodies or antibody fragments can be isolated from antibody phage libraries. High affinity (nM range) human antibodies can be generated by chain shuffling, as well as combinatorial infection and in vivo recombination as a strategy for constructing very large phage libraries. Thus, these techniques are viable alternatives to traditional monoclonal antibody hybridoma techniques for isolation of monoclonal antibodies.

The DNA that encodes the antibody may be modified to produce chimeric or fusion antibody polypeptides. The monoclonal antibodies used herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

Human and Humanized Antibodies

The anti-VEGF-D, anti-VEGFR-3-mediated signaling or anti-lymphangiogenesis antibodies used in the invention may comprise humanized antibodies or human antibodies. Generally, a “humanized antibody” is an antibody that has been modified using recombinant DNA techniques to circumvent the problem of a human's immune system reacting to an antibody as a foreign antigen. The standard procedure of producing monoclonal antibodies produces mouse antibodies. Although murine antibodies are very similar to human ones, there are differences. Consequently, the human immune system recognizes mouse antibodies as foreign, rapidly removing them from circulation and causing systemic inflammatory effects. “Humanized” forms of non-human (e.g., rodent) antibodies are chimeric antibodies that contain a reduced percentage of sequence derived from the non-human antibody. Various forms of humanized anti-VEGF-D, anti-VEGFR-3-mediated signaling or anti-lymphangiogenesis antibodies are contemplated. Humanized antibodies may be intact antibodies, such as intact IgG₁ antibodies, antibody chains or fragments thereof (such as Fv, Fab, Fab′, F(ab′)₂, or other antigen-binding subsequences of antibodies). Humanized antibodies include human antibodies (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human antibody are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human antibody and all or substantially all of the FR regions are those of a human antibody consensus sequence. The humanized antibody optimally also will comprise at least a portion of an antibody constant region (Fc), typically that of a human antibody.

Various humanization strategies have been described in the prior art and it is envisaged that practice of the invention extends to the use of both known humanization strategies and any new strategies to be developed in the future. Examples of known humanization strategies include those described by Studnicka (U.S. Pat. No. 5,869,619) and Padlan (1991, Molec. Immunol., 28, 489-498), Winter (U.S. Pat. No. 5,225,539) and Jones et al (1986, Nature, 321, 522-525), Queen et al. (U.S. Pat. No. 5,693,761) and Foote (U.S. Pat. No. 6,881,557).

As an alternative to humanization, human antibodies can be generated. For example, it is now possible to produce transgenic animals (e.g., mice) that are capable, upon immunization, of producing a full repertoire of human antibodies in the absence of endogenous immunoglobulin production.

Alternatively, phage display technology can be used to produce human antibodies and antibody fragments in vitro, from immunoglobulin variable (V) domain gene repertoires from unimmunized donors. Phage display can be performed in a variety of formats. Several sources of V-gene segments can be used for phage display.

Antibody Fragments.

“Antibody fragments” comprise a portion of an antibody, preferably the antigen binding or variable region of the intact antibody. Examples of antibody fragments include Fab, Fab′, F(ab′)₂, and Fv fragments; diabodies; linear antibodies; single chain antibody molecules; and multispecific antibodies formed from antibody fragments.

Papain digestion of antibodies produces two identical antigen binding fragments, called “Fab” fragments, and a residual “Fc” fragment, a designation reflecting the ability to crystallize readily. Each Fab fragment is monovalent with respect to antigen binding, i.e., it has a single antigen binding site. Pepsin treatment of an antibody yields a single large F(ab′)₂ fragment which roughly corresponds to two disulfide linked Fab fragments having divalent antigen binding activity and is still capable of cross linking antigen. Fab′ fragments differ from Fab fragments by having additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen recognition binding site. This fragment consists of a dimer of one heavy and one light chain variable region domain in tight, non covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single chain Fv” abbreviated as “scFv” are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding.

In certain circumstances there are advantages of using antibody fragments, rather than whole antibodies. The smaller size of the fragments allows for rapid clearance from the circulation.

Various techniques have been developed for the production of antibody fragments. Traditionally, these fragments were derived via proteolytic digestion of intact antibodies. However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments. According to another approach, F(ab′)₂ fragments can be isolated directly from recombinant host cell culture. Fab and F(ab′)₂ fragment with increased in vivo half-life comprising a salvage receptor binding epitope residues also may be used.

Other techniques for the production of antibody fragments will be apparent to the skilled practitioner. The antibody of choice is a single chain Fv fragment (scFv). Fv and scFv are the only species with intact combining sites that are devoid of constant regions; thus, they are suitable for reduced nonspecific binding during in vivo use. The antibody fragment may also be a “linear antibody”, which may be monospecific or bispecific.

Bispecific Antibodies

Bispecific antibodies are antibodies that have binding specificities for at least two different epitopes. Bispecific antibodies can be prepared as full length antibodies or antibody fragments (e.g., F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities.

According to a different approach, antibody variable domains with the desired binding specificity (antibody-antigen combining sites) are fused to immunoglobulin constant domain sequences.

Bispecific antibodies include cross-linked or “heteroconjugate” antibodies. Heteroconjugate antibodies are composed of two covalently joined antibodies. It is contemplated that the antibodies may be prepared in vitro using known methods in synthetic protein chemistry, including those involving crosslinking agents. For example, one of the antibodies in the heteroconjugate can be coupled to avidin, the other to biotin. Heteroconjugate antibodies may be made using any convenient cross-linking methods. Suitable cross-linking agents and cross-linking techniques are well known in the art.

Techniques for generating bispecific antibodies from antibody fragments have also been described in the literature. For example, bispecific antibodies can be prepared using chemical linkage.

Recent progress has facilitated the direct recovery of Fab′-SH fragments from E. coli, which can be chemically coupled to form bispecific antibodies. Various techniques for making and isolating bispecific antibody fragments directly from recombinant cell culture have also been described.

The term “diabodies” refers to small antibody fragments prepared by constructing scFv fragments with short linkers (about 5 to 10 residues) between the VH and VL domains such that inter chain but not intra chain pairing of the V domains is achieved, resulting in a bivalent fragment, i.e., fragment having two antigen binding sites. Bispecific diabodies are heterodimers of two “crossover” scFv fragments in which the VH and VL domains of the two antibodies are present on different polypeptide chains.

According to an alternative “diabody” technology for making bispecific antibody fragments, the fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported.

Antibodies with more than two valencies are contemplated for use in the invention. For example, trispecific antibodies can be prepared.

Multivalent Antibodies

A multivalent antibody may be internalized (and/or catabolized) faster than a bivalent antibody by a cell expressing an antigen to which the antibodies bind.

Antibodies that may be used in the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. A preferred dimerization domain comprises an Fc region or a hinge region. In this scenario, the antibody will comprise an Fc region and three or more antigen binding sites amino-terminal to the Fc region. A preferred multivalent antibody comprises three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X₁)ₙ-VD2-(X₂)ₙ-Fc, wherein VD1 is a first variable domain, VD2 is a second variable domain, Fc is one polypeptide chain of an Fc region, X₁ and X₂ represent an amino acid or polypeptide, and n is 0 or 1. For instance, the polypeptide chain(s) may comprise: V_{H}-C_{H}1-flexible linker-V_{H}-C_{H}1-Fc region chain; or V_{H}-C_{H}1-V_{H}-C_{H}1-Fc region chain. A multivalent antibody preferably further comprises at least two (and preferably four) light chain variable domain polypeptides. A multivalent antibody may, for instance, comprise from about two to about eight light chain variable domain polypeptides. The light chain variable domain polypeptides contemplated here comprise a light chain variable domain and, optionally, further comprise a CL domain.

Peptide and Peptide Mimetic Inhibitors

In another embodiment, the inhibitor of VEGF-D activity, VEGFR-3-mediated signaling or lymphangiogenesis is a peptide or peptide mimetic. The peptide or peptide mimetic may reduce receptor availability for native ligand binding.

As used herein, “peptide mimetic” and “peptidomimetic” are used interchangeably.

A peptide inhibitor is a peptide that binds specifically to a component of VEGF-D activity, VEGFR-3-mediated signaling or lymphangiogenesis and inhibits or neutralizes the function of that component in the process of VEGF-D activity, VEGFR-3-mediated signaling or lymphangiogenesis. Peptide inhibitors may be chemically synthesized using known peptide synthesis methodology or may be prepared and purified using recombinant technology. The preferred length of peptide inhibitors of VEGF-D activity, VEGFR-3-mediated signaling or lymphangiogenesis is from about 6, 7, 8, 9 or 10 amino acid residues to about 100 amino acid residues. It is contemplated that longer peptides may prove useful. Peptide inhibitors may be identified without undue experimentation using well known techniques. In this regard, it is noted that techniques for screening peptide libraries for peptides that are capable of specifically binding to a polypeptide target are well known in the art.

For any of the foregoing peptides, one preferred variation involves peptides that have been modified to comprise an intramolecular bond between two non-adjacent amino acid residues of the primary sequence, thereby forming a cyclic peptide. For example, in one variation, the peptide comprises a pair of cysteine residues, such as amino- and carboxy-terminal cysteines, and the intramolecular bond comprises a disulfide bond between the cysteines. However, organic chemists and peptide chemists are capable of synthesizing intramolecular bonds between a wide variety of amino acids using conventional techniques.

Nucleic Acid Molecules

Antisense Molecules

In yet another embodiment, the inhibitor of VEGF-D activity, VEGFR-3-mediated signaling or lymphangiogenesis is an antisense molecule that reduces transcription and/or translation of a component of VEGF-D activity, VEGFR-3-mediated signaling or lymphangiogenesis, thereby reducing VEGF-D activity, VEGFR-3-mediated signaling or lymphangiogenesis.

The antisense molecule comprises RNA or DNA prepared using antisense technology, where, for example, an antisense RNA or DNA molecule acts to block directly the translation of mRNA by hybridizing to targeted mRNA and preventing protein translation. Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to reduce or block expression of a component of VEGF-D activity, VEGFR-3-mediated signaling or lymphangiogenesis, and thus VEGF-D activity, VEGFR-3-mediated signaling or lymphangiogenesis. Such oligonucleotides can also be delivered to cells such that the antisense RNA or DNA may be expressed in vivo to inhibit production of components of VEGF-D activity, VEGFR-3-mediated signaling or lymphangiogenesis.

Inhibitors of VEGF-D activity or signaling mediated by VEGFR-3, or lymphangiogenesis include antisense or sense oligonucleotides comprising a single-stranded nucleic acid sequence (either RNA or DNA) capable of binding to target mRNA (sense) or DNA (antisense) sequences. Such a fragment generally comprises about 10 to 40 nucleotides in length, preferably at least about 14 nucleotides, preferably from about 14 to 30 nucleotides.

Antisense or sense oligonucleotides further comprise oligonucleotides having modified sugar-phosphodiester backbones that are resistant to endogenous nucleases, or are covalently linked to other moieties that increases affinity of the oligonucleotide for a target nucleic acid sequence, or intercalating agents to modify binding specificities of the antisense or sense oligonucleotide for the target nucleotide sequence.

Small Interfering RNA (siRNA)

In one embodiment, it is envisaged that siRNA will inhibit VEGF-D activity, VEGFR-3-mediated signaling or lymphangiogenesis. “siRNA” or “RNAi” are double-stranded RNA molecules, typically about 21 nucleotides in length, that are homologous to a gene or polynucleotide that encodes the target gene and interfere with the target gene's expression.

Nucleic Acid Molecules in Triple-Helix Formation

In another embodiment, the inhibitor of VEGF-D activity, VEGFR-3-mediated signaling or lymphangiogenesis comprises nucleic acid molecules in triple-helix formation. Nucleic acid molecules in triple-helix formation used to inhibit transcription should be single-stranded and composed of deoxynucleotides. A DNA oligonucleotide is designed to be complementary to a region of the gene involved in transcription. The base composition of these oligonucleotides is designed such that it promotes triple-helix formation via Hoogsteen base-pairing rules, which generally require sizeable stretches of purines or pyrimidines on one strand of a duplex.

Ribozymes

In a related embodiment, the inhibitor of VEGF-D activity, VEGFR-3-mediated signaling or lymphangiogenesis is a ribozyme that reduces transcription of a component of VEGF-D activity or signaling mediated by VEGFR-3, or a lymphangiogenic component.

A “ribozyme” is an enzymatic RNA molecule capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques.

Small Molecule Inhibitors

In a further embodiment, the inhibitor of VEGF-D activity, VEGFR-3-mediated signaling or lymphangiogenesis is a small molecule.

A “small molecule” is defined herein to have a molecular weight below about 2000 daltons, and preferably below about 500 Daltons. Potential inhibitors of VEGF-D activity, VEGFR-3-mediated signaling or lymphangiogenesis include small molecules that bind to the active site, the receptor binding site, or growth factor or other relevant binding site of components of VEGF-D activity or VEGFR-3-mediated signaling, or lymphangiogenesis, thereby blocking the normal biological activity of VEGF-D, VEGFR-3-mediated signaling or lymphangiogenesis. Examples of small molecules include, but are not limited to, synthetic non-peptidyl organic or inorganic compounds.

Small molecule inhibitors of VEGF-D activity, VEGFR-3-mediated signaling or lymphangiogenesis may be identified without undue experimentation using known techniques and chemically synthesized using known methodology. In this regard, it is noted that techniques for screening organic molecule libraries for molecules that are capable of binding to a polypeptide target are known in the art.

Inhibition of Receptor Availability for Ligand Binding

Antibody Inhibitors

In one embodiment, the inhibitor of VEGF-D activity, VEGFR-3-mediated signaling or lymphangiogenesis is an antibody. In a preferred embodiment, the inhibitor of VEGF-D activity, VEGFR-3-mediated signaling or lymphangiogenesis is an anti-VEGFR-3 antibody that reduces VEGFR-3 availability for ligand binding.

Suitable antibodies for use in the methods of the invention and means for their production are disclosed in WO2000/021560 and WO1995/021868 and include a polyclonal or a monoclonal antibody that binds specifically to VEGFR-3 and blocks its signaling, a fragment of such an antibody, a chimeric antibody, a humanized antibody, and a bispecific antibody that binds specifically to VEGFR-3 and blocks its signaling and also binds to another antigen.

In a preferred embodiment, the antibody inhibitor is a humanized antibody. In another embodiment, the antibody inhibitor of VEGF-D activity, VEGFR-3-mediated signaling or lymphangiogenesis comprises a Fab, Fab′, or F(ab′)₂ fragment, or a single chain Fv (scFv) fragment.

Persons skilled in the art will appreciate that in particular embodiments, the monoclonal antibody may comprise antibody 9D9F9, disclosed in WO2000/021560 or 2E11D11 disclosed in WO2003/006104. Alternatively monoclonal antibodies that specifically bind to VEGFR-3 and may be used according to the invention include antibodies MM0003-7G63, RM0003-5F63, C28G5, KLT9, ZMD.251, mF4-31C1 and hF4-3C5. A particularly preferred monoclonal antibody is hF4-3C5, a fully-humanized antagonist antibody to human VEGFR-3.

In an alternative embodiment, the inhibitor may comprise a bispecific antibody, particularly a diabody, that binds specifically to and neutralizes each of VEGFR-3 and a second target. One example of such a diabody is that derived from antibodies hF4-3C5 and IMC-1121, which binds specifically to and neutralizes each of VEGFR-3 and VEGFR-2.

An inhibitor of VEGF-D activity, VEGFR-3-mediated signaling or lymphangiogenesis according to the present invention also includes in one embodiment an antibody, as described above, that inhibits or neutralizes the receptor tyrosine kinase activity of VEGFR-3.

Peptide and Peptide Mimetic Inhibitors

The person skilled in the art will appreciate that particular inhibitors of VEGF-D activity, VEGFR-3-mediated signaling or lymphangiogenesis that can be employed in a particular embodiment of the present invention are disclosed in WO2000/021560, WO2001/052875, and WO2002/057299, which are incorporated herein by reference. In one embodiment, the inhibitor of VEGF-D activity, VEGFR-3-mediated signaling or lymphangiogenesis comprises a peptide. Such a peptide to be used as an inhibitor of VEGF-D activity, VEGFR-3-mediated signaling or lymphangiogenesis can be generated by random peptide synthesis, by recombinant means from random oligonucleotides, or a peptide may be selected from a phage display library, according to the disclosure of WO2002/057299 and WO2000/021560 and methods standard in the art. Such a peptide can be identified with the aid of the VEGFR-3 extracellular domain.

In a particular embodiment, the peptide inhibitor of VEGF-D activity, VEGFR-3-mediated signaling or lymphangiogenesis comprises the amino acid sequence GYWX₁X₂X₃W, wherein X₁, X₂, and X₃ comprise amino acids and wherein the peptide binds VEGFR-3, according to WO2002/057299. In a related embodiment, the peptide inhibitor comprises the amino acid sequence GYWX₁X₂X₃WX₄, wherein X₄ comprises an amino acid. In another embodiment, either of the preceding peptides may further comprise an amino- and carboxy-terminus cysteine residue. In a particular embodiment, the peptide comprises a cyclic peptide. In an alternative embodiment, the peptide comprises a peptide dimer that binds to VEGFR-3, and in a preferred form, the peptides comprising the dimer are the same, according to WO2002/057299.

In one embodiment, the peptidomimetic inhibitor is a monomeric monocyclic peptide inhibitor or dimeric bicyclic peptide inhibitor. Preferably, such peptidomimetic inhibitors are based on the peptide sequence of exposed loops of growth factor proteins, for example, loops 1, 2, and 3 of VEGF-D. In a preferred embodiment, the peptidomimetic inhibitor comprises any one of: CASELGKSTNTFC; CNEESLIC; or CISVPLTSVPC.

In one embodiment, the peptide mimetic inhibitor is prepared by the methods disclosed in WO2001/052875 and WO2002/057299. Peptides that may be used as inhibitors of VEGF-D activity, VEGFR-3-mediated signaling or lymphangiogenesis are disclosed in WO2000/021560. Such peptides include a polypeptide comprising a fragment or analog of a vertebrate VEGF-C polypeptide, wherein the polypeptide and fragment or analog are capable of binding to VEGFR-3, but do not activate signaling, and a polypeptide comprising a fragment or analog of a vertebrate VEGF-D polypeptide, wherein the polypeptide and fragment or analog are capable of binding to VEGFR-3, but do not activate signaling.

The person skilled in the art will appreciate that inhibitors of VEGF-D activity, VEGFR-3-mediated signaling or lymphangiogenesis inhibitors according to WO2002/057299 include peptides comprising the sequence YIGYWLTIWGY₂, wherein Y, and Y₂ are amino acids. In one variation, the peptide is made cyclic by a bond between Y and Y₂. In a specific preferred embodiment, the peptide comprises the sequence CGYWLTIWGC. Other peptide inhibitors comprise any of the following amino acid sequences: SGYWWDTWF, SCYWRDTWF, KVGWSSPDW, FVGWTKVLG, YSSSMRWRH, RWRGNAYPG, SAVFRGRWL, WFSASLRFR, WQLGRNWI, VEVQITQE, AGKASSLW, RALDSALA, YGFEAAW, YGFLWGM, SRWRILG, HKWQKRQ, MDPWGGW, RKVWDIR, VWDHGV, CWQLGRNWIC, CVEVQITQEC, CAGKASSLWC, CRALDSALAC, CYGFEAAWC, CYGFLWGMC, CSRWRILGC, CHKWQKRQC, CMDPWGGWC, CRKVWDIRC, CVWDHGVC, CGQMCTVWCSSGC, or conservative substitutions-variants thereof. Preferred peptides comprise these exact amino acid sequences, or sequences in which only one or only two conserved substitutions have been introduced. In another preferred variation, the peptides comprise amino- and carboxy-terminal cysteines, which permit formation of cyclic molecules and dimers and multimers. In yet another variation, peptide inhibitors include the amino acid sequence GYWXIX₂X₃W, wherein X, X₂, and X₃ comprise amino acids, the amino acid sequence GYWX, XZX₃WX₄, wherein X₄ comprises an amino acid. In still another variation, these peptides further comprise amino- and carboxy-terminal cysteine residues.

Nucleic Acid Inhibitors

In a preferred embodiment, the invention envisages use of a VEGFR-3 antisense RNA, as disclosed in WO2000/021560, to inhibit the translation of VEGFR-3-encoding mRNA to eliminate or downregulate levels of VEGFR-3. Similarly, siRNA or nucleic acids in triple helix formation could be used to reduce VEGFR-3 availability for ligand binding.

Small Molecule Inhibitors

In a preferred embodiment, the small molecule is a small molecule inhibitor of receptor tyrosine kinase activity. In a more preferred embodiment, the small molecule comprises PTK787/ZK22854, AZP2171, ZK991, KRN633, MAZ51, sorafenib, sunitinib (SU11248), axitinib (AG013736), vandetanib (ZD6474), or 3-(indole-3-yl)-4-(3,4,5-trimethoxyphenyl)-1H-pyrrole-2,5-dione.

Inhibition of Ligand Availability for Receptor Binding

Antibody Inhibitors

According to one embodiment, inhibition of VEGF-D activity, VEGFR-3-mediated signaling or lymphangiogenesis can be achieved using antibodies that specifically bind and neutralize ligands for VEGFR-3, that is, VEGF-C and/or VEGF-D. Antibodies similar to anti-VEGFR-3 antibodies described above are contemplated. Suitable antibodies and their means for production are disclosed in WO2000/021560. The person skilled in the art will appreciate that antibodies that bind specifically to VEGF-D and may be used according to the invention include monoclonal antibodies 2F8, 4A5 (also known as VD1), 4E10, 5F12, 4H4 and 3C10 disclosed in WO2000/037025. A particularly preferred antibody is 4A5, and in particular, a humanized version thereof. In another embodiment, the chimeric or humanized antibody comprises SEQ ID NO: 37 and SEQ ID NO: 39, or the antibody comprises any one of SEQ ID NOs: 47 to 49 and any one of SEQ ID NOs: 50 to 52, as disclosed in WO2005/087177. Alternatively monoclonal antibodies that may be used according to the invention include 28AT743.288.48, MM0007-7E79, RM0007-8C35, 78902, 78923, 78939, and 90409.

Similarly, monoclonal antibodies that bind VEGF-C may be employed. Suitable examples of such antibodies include antibodies 103, MM0006-2E65 and 193208. Further examples of such antibodies are found in U.S. Pat. No. 7,208,582 and U.S. Pat. No. 7,109,308. A particularly preferred antibody is V069D09.

Alternatively, antibodies may bind proprotein convertases, enzymes responsible for processing VEGF-C and VEGF-D from their prepro-forms to their activated forms, and reduce, inhibit or neutralize such activity thereby limiting the amount of proteolytically processed ligand available for binding to VEGFR-3. Again, antibodies corresponding with anti-VEGFR-3 antibodies described above are envisaged. Such antibodies are disclosed in WO05/112971 and include neutralizing antibodies to inhibit the biological action of proprotein convertases.

Peptide Inhibitors

Inhibitors of VEGF-D activity, VEGFR-3-mediated signaling or lymphangiogenesis as used in the present invention include inhibitors of proprotein convertases. As noted, one class of inhibitor of proprotein convertases comprises antibodies. Another class of inhibitor of proprotein convertases includes peptide inhibitors.

Peptide inhibitors of proprotein convertases are disclosed in WO05/112971 and include prosegments of proprotein convertases, inhibitory variants of anti-trypsin and peptidyl haloalkylketone inhibitors.

Representative inhibitory prosegments of proprotein convertases include the inhibitory prosegments of PC5A (also known as PC6A), PC5B (also known as PC6B), PACE4, PC1 (also known as PC3), PC2, PC4, PC7 and Furin. A representative inhibitory variant of anti-trypsin is α-1 antitrypsin Portland, an engineered variant of naturally occurring antitrypsin that inhibits multiple proprotein convertases. Representative peptidyl halomethyl ketone inhibitors include decanoyl-Arg-Val-Lys-Arg-chloromethylketone (Dec-RVKR-CMK), decanoyl-Phe-Ala-Lys-Arg-chloromethylketone (Dec-FAKR-CMK), decanoyl-Arg-Glu-Ile-Arg-chloromethylketone (Dec-REIR-CMK), and decanoyl-Arg-Glu-Lys-Arg-chloromethylketone (Dec-REKR-CMK). These inhibitors of proprotein convertases, such as Dec-RVKR-CMK or the inhibitory prosegments of proprotein convertases, can be used to block the activation of VEGF-C and VEGF-D and thereby inhibit VEGF-D activity, VEGFR-3-mediated signaling or lymphangiogenesis induced by partially processed or fully processed VEGF-C or VEGF-D.

Soluble Receptors

According to another embodiment, VEGF-D activity, VEGFR-3-mediated signaling or lymphangiogenesis can be inhibited using soluble receptors that bind VEGFR-3 ligands. Soluble receptors capable of binding VEGF-C and VEGF-D, thereby inhibiting VEGF-D activity or signaling via VEGFR-3, are disclosed in WO2000/023565, WO2000/021560 and WO2002/060950. Such inhibitors of VEGF-D activity, VEGFR-3-mediated signaling or lymphangiogenesis inhibitors include soluble VEGFR-2, VEGFR-3, NRP-1, and NRP-2.

Nucleic Acid Inhibitors

In another embodiment of the invention, antisense oligonucleotides are used as inhibitors of proprotein convertases. The antisense oligonucleotides preferably inhibit expression of proprotein convertases by inhibiting transcription or translation of proprotein convertases. In a further embodiment, the antagonizing agent is small interfering RNAs (siRNA, also known as RNAi, RNA interference nucleic acids). Also contemplated are methods of inhibiting the target gene expression or target protein function utilizing ribozymes and triplex-forming nucleic acid molecules.

Similarly, in a related embodiment, antisense, siRNA and ribozyme inhibitors directed to VEGF-C and/or VEGF-D are included as inhibitors of VEGF-D activity, VEGFR-3-mediated signaling or lymphangiogenesis exerting their effects by reducing transcription and/or translation of VEGF-C and VEGF-D.

Peptide and Peptide Mimetic Inhibitors

According to one embodiment, the inhibitor to be used in the invention comprises a peptide that reduces the availability of ligand to bind to VEGFR-3. Such a peptide can be generated by random peptide synthesis, by recombinant means from random oligonucleotides, or a peptide may be selected from a phage display library by methods standard in the art. In a particular embodiment, the peptide will be derived from VEGFR-3 or VEGFR-2 and will bind specifically to VEGF-C or VEGF-D such that the ligand available for binding to native VEGFR-3 is reduced. Such a peptide may be identified with the aid of the VEGF-C or VEGF-D.

Small Molecule Inhibitors

In one embodiment, the small molecule inhibitor is a small molecule inhibitor of a proprotein convertase. In a particular embodiment, the proprotein convertase is furin and the small molecule comprises B3 (CCG8294, naphthofluorescein disodium) or a derivative of 2,5-dideoxystreptamine.

Antibody Inhibitors Affecting Ligand-Receptor Complex

In one embodiment, the invention includes use of bispecific antibodies, as described above, as inhibitors of VEGF-D activity, VEGFR-3-mediated signaling or lymphangiogenesis, specifically inhibiting ligand-receptor complexes.

Suitable antibodies and their means for production are disclosed in WO2000/021560 and include a bispecific antibody that binds specifically to an epitope or epitopes derived from a VEGFR-3-(VEGFR-3 ligand) complex (receptor-ligand complex) and blocks VEGFR-3 signaling.

Inhibition of Co-Receptor Interaction

Antibody Inhibitors Affecting Co-Receptors of VEGFR-3

In a further embodiment, inhibitors of VEGF-D activity, VEGFR-3-mediated signaling or lymphangiogenesis include antibodies, as described above, that bind specifically to and reduce, inhibit or neutralize co-receptor binding to VEGFR-3. Such antibodies may be directed to a co-receptor, a ligand-co-receptor binary complex, a co-receptor-receptor binary complex, or a ligand-co-receptor-receptor ternary complex. Co-receptors include NRP-1 and NRP-2. The person skilled in the art will understand that monoclonal antibodies that specifically bind NRP-1 or NRP-2 and may be used according to the invention include antibodies 1B3, 3G6-2C5, AD5-17F6, 446915, 446921, 130603, 130604, 96009, 3B8, 54, 257103, 257107, A-12, and C-9. Alternatively, a bispecific antibody which specifically binds to NRP-2 receptor and a VEGF-C polypeptide, as disclosed in WO2003/029814, may be used according to the invention.

Peptide Inhibitors Affecting Co-Receptors of VEGFR-3

In another embodiment, a peptide inhibitor comprising a peptide dimer may target one or more receptors and/or co-receptors. Co-receptors include NRP-1 and NRP-2. As disclosed in WO2002/057299, in a particular embodiment, the peptide dimer comprises one peptide that binds VEGFR-3 and a second peptide that binds to any one of VEGFR-1, VEGFR-2, NRP-1, or NRP-2.

Small Molecule and Nucleic Acid Inhibitors Affecting Co-Receptors of VEGFR-3

According to the present invention, it is also envisaged that small molecules, antisense molecules, siRNA and ribozymes, as described above, can be utilized as inhibitors of VEGF-D activity, VEGFR-3-mediated signaling or lymphangiogenesis by targeting co-receptors that interact with VEGFR-3. Such co-receptors include NRP-1 and NRP-2.

Inhibition of Downstream Signaling

Alternatively, an inhibitor of VEGF-D activity, VEGFR-3-mediated signaling or lymphangiogenesis according to any of the foregoing descriptions may disrupt downstream intracellular VEGFR-3 signaling, as disclosed in WO2000/021560.

Other Inhibitors

One skilled in the art will appreciate that many of the foregoing embodiments will be suitable for inhibition of VEGF-D activity, VEGFR-3-mediated signaling or lymphangiogenesis by their application to NRP-1, NRP-2, ANG-1, TIE-2 and/or integrins. In one embodiment, inhibition of VEGF-D activity, VEGFR-3-mediated signaling or lymphangiogenesis is mediated by inhibition of integrin α9α1. Monoclonal antibodies that may be used according to the invention include antibodies 171718, 3A5, PADZMD.572, 1E11DH, 4G8HE, MM0018-21G7, 171733, and Y9A2.

Screening for Inhibitors

To assay for inhibitors of VEGF-D activity, VEGFR-3-mediated signaling or lymphangiogenesis, VEGF-C or VEGF-D may be added to a cell expressing VEGFR-3 along with the candidate compound to be screened for inhibitor activity, and the ability determined of the compound to inhibit endothelial cell migration, endothelial cell proliferation, or phosphorylation of VEGFR-3. Inhibition of any of these activities in the presence of VEGF-D indicates that the candidate compound is an inhibitor of VEGF-D activity, VEGFR-3-mediated signaling or lymphangiogenesis. Inhibition of any of these activities in the presence of VEGF-C indicates that the candidate compound is an inhibitor of VEGFR-3-mediated signaling or lymphangiogenesis. Alternatively, inhibitors may be detected by combining VEGF-C or VEGF-D and a potential inhibitor with VEGFR-3 under appropriate conditions for a competitive inhibition assay. The VEGF-C or VEGF-D polypeptide can be labeled, such as by radioactivity, such that the number of VEGF-C or VEGF-D polypeptide molecules bound to VEGFR-3 can be used to determine the effectiveness of the potential inhibitor.

In another assay for inhibitors, mammalian cells or a membrane preparation expressing VEGFR-3 would be incubated with a labeled VEGF-C or VEGF-D polypeptide in the presence of the candidate compound. The ability of the compound to enhance or block this interaction could then be measured.

Small molecule inhibitors of VEGF-D activity, VEGFR-3-mediated signaling or lymphangiogenesis are identifiable by standard in vitro screening assays, e.g. using VEGF-D and recombinantly expressed VEGFR-3, and are also disclosed in WO2000/021560.

The skilled person will be able to conduct similar assays to screen for inhibitors of proprotein convertases, NRP-1, NRP-2, ANG-1, TIE-2, or integrins, including integrin α9β1.

Formulation for Administration

The inhibitor of VEGF-D activity, VEGFR-3-mediated signaling or lymphangiogenesis may be provided as a pharmaceutical or veterinary composition.

A pharmaceutical composition is one which is suitable for administration to humans. A veterinary composition is one that is suitable for administration to animals.

The pharmaceutical or veterinary compositions used in the methods of the invention may comprise one or more pharmaceutically acceptable carriers and optionally other therapeutic agents. Each carrier, diluent, adjuvant and/or excipient must be pharmaceutically “acceptable”.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained. Similarly, a “pharmaceutically acceptable” salt or ester of an inhibitor of VEGF-D activity, VEGFR-3-mediated signaling or lymphangiogenesis is a salt or ester which is not biologically or otherwise undesirable.

As used herein, a “pharmaceutical carrier” is a pharmaceutically acceptable solvent, suspending agent or vehicle for delivering the agent to the subject. The carrier may be liquid or solid and is selected with the planned manner of administration in mind. Each carrier must be pharmaceutically “acceptable” in the sense of being not biologically or otherwise undesirable i.e. the carrier may be administered to a subject along with the agent without causing any or a substantial adverse reaction.

The pharmaceutical composition may be administered orally, intranasally, topically, or parenterally in formulations containing conventional non-toxic pharmaceutically acceptable carriers, adjuvants, and vehicles.

The term parenteral as used herein includes intravenous, intraarterial, intraperitoneal, intramuscular, subcutaneous, subconjunctival, intracavity, and transdermal injection, aerosol for administration to lungs or nasal cavity or administration by infusion by, for example, osmotic pump.

Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or antibodies; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The compound or composition may be administered orally as tablets, aqueous or oily suspensions, lozenges, troches, powders, granules, emulsions, capsules, syrups or elixirs.

The composition for oral use may contain, in order to produce pharmaceutically elegant and palatable preparations, one or more agents or excipients selected from the group of: (1) inert diluents, such as calcium carbonate, lactose, calcium phosphate or sodium phosphate; (2) granulating and disintegrating agents, including corn starch, methylcellulose, polyvinylpyrrolidone, xanthan gum, bentonite, alginic acid or agar; (3) binding agents, such as starch, gelatin or acacia; (4) lubricating agents, such as magnesium stearate, stearic acid sodium oleate, talc or sodium chloride; (5) sweetening agents, such as sucrose, lactose, glucose, aspartame or saccharin; (6) flavoring agents, including peppermint oil, oil of wintergreen, cherry, orange or raspberry flavoring; (7) coloring agents; (8) time delay agents including glyceryl monostearate or glyceryl distearate; and (9) preserving agents, including sodium benzoate, vitamin E, alpha-tocopherol, ascorbic acid, methyl paraben, propyl paraben or sodium bisulphite.

Sustained-release compositions may be prepared. For example, tablets may be uncoated, or coated by techniques known to delay disintegration and absorption in the gastrointestinal tract and thereby provide a sustained action over a longer period. Suitable examples of sustained-release compositions include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Where sustained-release administration of an inhibitor of VEGF-D activity, VEGFR-3-mediated signaling or lymphangiogenesis is desired in a composition with release characteristics suitable for the treatment of pulmonary edema requiring administration of the inhibitor, microencapsulation of the inhibitor is contemplated. The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Microencapsulation of recombinant proteins for sustained release has been successfully performed with human growth hormone (rhGH), interferon- (rhIFN-), interleukin-2, and MN rgp120.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition.

The composition to be delivered orally may also be administered as a spray, mist or fine dry powder emitted from a suitable delivery device such as a nebulizer etc.

The composition may also be delivered intranasally using a suitable delivery device such as nebulizer.

For pulmonary administration, the composition, e.g. an antibody composition, may be formulated for and suitably delivered in a particle size effective for reaching the lower airways of the lung. Any of a variety of inhalation or nasal delivery devices known in the art for administration of a therapeutic agent may be employed. Such devices are capable of depositing aerosolized formulations in the alveoli of a patient and include metered dose inhalers, nebulizers, dry powder generators, sprayers, and the like. Such devices dispense the formulation as an aerosol. Aerosols can be comprised of solutions (aqueous or non-aqueous), suspensions or solid particles. Some nebulizers produce aerosols from solutions, whilst metered dose inhalers, dry powder inhalers, etc. generate small particle aerosols.

There are several desirable features of an inhalation device for administering the active agent, such as an antibody. For example, delivery by the inhalation device is advantageously reliable, reproducible and accurate.

Formulations suitable for use with a sprayer or nebulizer may include, for example, the active ingredient (i.e. the inhibitor) in an aqueous solution at a concentration of about 0.1 mg to about 100 mg per milliliter. The formulation can include agents such as an excipient, an isotonicity agent, a preservative, a surfactant, and, preferably, zinc. The formulation can also include an excipient or agent for stabilization of the antibody composition, such as a buffer, a reducing agent, a bulk protein, or a carbohydrate. Bulk proteins useful in formulating such compositions include albumin, protamine, or the like. Typical carbohydrates useful in formulating the compositions include sucrose, mannitol, lactose, trehalose, glucose, or the like.

The surfactant can reduce or prevent surface-induced aggregation of the active agent, such as an antibody, caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan mono-oleate, polysorbate 80, polysorbate 20, or the like. The surfactant can be chosen to stabilize the active agent as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol.

Advantageously, the inhalation device can deliver particles of size less than about 10 μm, preferably in the range of about 1 μm to about 5 μm, and most preferably about 2 μm to about 3 μm, for good respirability.

A formulated spray including the composition can be produced by forcing a suspension or solution of the composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

The composition may also be delivered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of the composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation either directly or through a coupling fluid, creating an aerosol including the antibody composition protein.

Dry powder inhalers use breath-actuation of a mixed powder (see, e.g. U.S. Pat. No. 4,668,218, EP 237507, WO 97/25086, WO 94/08552, U.S. Pat. No. 5,458,135, WO 94/06498). Metered dose inhalers typically use a propellant gas and require actuation during inspiration (see, e.g., WO 94/16970, WO 98/35888). In a metered dose inhaler, a propellant, the active agent (i.e. the inhibitor) and any excipients or other additives are contained in a canister as a mixture including a liquefied compressed gas. Actuation of the metering valve releases the mixture as an aerosol. The desired aerosol particle size can be obtained by employing a formulation of the composition produced by various methods known to those of skill in the art, including jet-milling, spray drying, critical point condensation, or the like. Formulations for use with a metered dose inhaler will generally include a finely divided powder containing the active agent as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like. Preferably the propellant is a hydrofluorocarbon.

If the VEGF-D activity target, VEGFR-3-mediated signaling target or lymphangiogenesis target is intracellular and whole antibodies are used as inhibitors, internalizing antibodies are preferred. However, lipofections or liposomes can also be used to deliver the antibody, antibody fragment, or other inhibitor into cells. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Where antibody fragments are used, the smallest inhibitory fragment that specifically binds to the binding domain of the target protein is preferred. For example, based upon the variable-region sequences of an antibody, antibody fragments can be designed that retain the ability to bind the target protein sequence. Such antibody fragments can be synthesized chemically and/or produced by recombinant DNA technology.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The compositions may also contain other active compounds as necessary for the particular indication being treated, preferably those with complementary activities that do not adversely affect each other, providing supplemental, additional, or enhanced therapeutic functions. Alternatively, or additionally, the composition may comprise an agent that enhances the function of the active compound. Such other active compounds and/or agents are suitably present in the composition in amounts that are effective for the purpose intended.

Other therapeutically useful agents, such as growth factors (e.g., BMPs, TGF-P, FGF, IGF), cytokines (e.g., interleukins and CDFs), antibiotics, and any other therapeutic agent beneficial for the condition being treated, may optionally be included in or administered simultaneously or sequentially with the inhibitor of VEGF-D activity, VEGFR-3-mediated signaling or lymphangiogenesis. In one embodiment, inhibitors of VEGFR-2 signaling are administered in combination with the inhibitor of VEGF-D activity, VEGFR-3-mediated signaling or lymphangiogenesis.

Administration “in combination with” one or more further therapeutic agents includes simultaneous (concurrent) and consecutive administration in any order. In particular embodiments, the invention contemplates administration of an inhibitor as described herein in combination with a complementary inhibitor as known or described elsewhere in the literature. Alternatively, in a preferred embodiment, the invention includes administration of an inhibitor as described herein in combination with a therapeutic agent that targets the risk condition or trigger event that is associated with development of pulmonary edema. The second agent or medicament to be administered will depend upon the identity of the risk condition that is associated with development of pulmonary edema. In such an embodiment, the combination of inhibitor and agent will treat pulmonary edema and its underlying cause.

The inhibitor of VEGF-D activity, VEGFR-3-mediated signaling or lymphangiogenesis may also be presented for use in the form of veterinary compositions, which may be prepared, for example, by methods that are conventional in the art. Examples of such veterinary compositions include those adapted for:

(a) oral administration, external application, for example drenches (e.g. aqueous or non-aqueous solutions or suspensions); tablets or boluses; powders, granules or pellets for admixture with feed stuffs; pastes for application to the tongue, particularly adapted for protection through the rumen if to be administered to ruminants;

(b) parenteral administration for example by subcutaneous, intramuscular or intravenous injection, e.g. as a sterile solution or suspension; or (when appropriate) by intramammary injection where a suspension or solution is introduced in the udder via the teat;

(c) topical applications, e.g. as a cream, ointment or spray applied to the skin; or

(d) intravaginally, e.g. as a pessary, cream or foam.

It is especially advantageous to formulate the veterinary or pharmaceutical compositions in dosage unit form for ease of administration and uniformity of dosage. Dosage unit form as used herein refers to physically discrete units, such as a container, pack, or dispenser, suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Instructions for administration may also be included.

Dosages and desired drug concentrations of pharmaceutical compositions of the present invention may vary depending on the particular use envisioned. The determination of the appropriate dosage or route of administration is well within the skill of an ordinary physician.

When in vivo administration of an inhibitor of VEGF-D activity, VEGFR-3-mediated signaling or lymphangiogenesis is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature. It is anticipated that different formulations will be effective for different treatment compounds and different disorders, that administration targeting one organ or tissue, for example, may necessitate delivery in a manner different from that to another organ or tissue.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

All references, including any patents or patent application, cited in this specification are hereby incorporated by reference. No admission is made that any reference constitutes prior art. It will be clearly understood that, although a number of prior art publications are referred to herein, this reference does not constitute an admission that any of these documents form part of the common general knowledge in the art, in Australia or in any other country.

### EXAMPLES

The invention is now further described in detail by reference to the following examples. The examples are provided for purposes of illustration only, and are not intended to be limiting unless otherwise specified. Thus, the invention encompasses any and all variations which become evident as a result of the teaching provided herein.

### Example 1

Previously, the inventors generated a mutant mouse via gene targeting that does not express VEGF-D (Vegfd knockout mouse). These mice are viable, fertile and have a relatively normal lymphatic system, although the absolute abundance of lymphatic vessels in the lung is reduced by approximately 30% compared to wild-type mice. In light of this reduction, the inventors conducted hyperoxia experiments, designed to induce pulmonary edema, to test the functional properties of the pulmonary lymphatic vasculature in these mice.

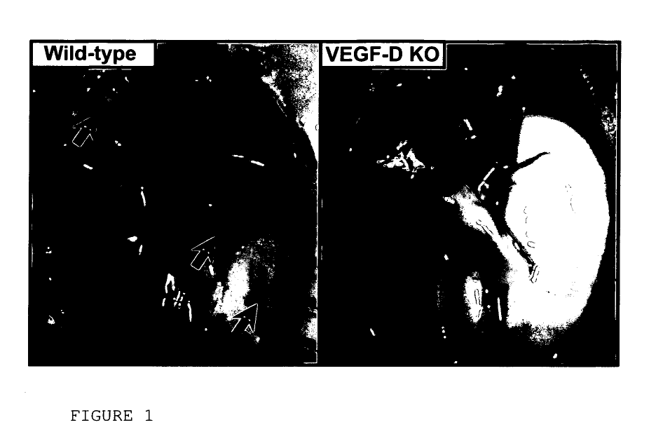

Male Vegfd knockout (7-10 weeks old on C57BL/6 genetic background) and wild-type control mice were exposed to hyperoxic conditions (approximately 95% O₂ for 60 hours), designed to induce pulmonary edema, then returned to normoxia for 24 hours so the rate at which the edema resolved could be determined. Unexpectedly, macroscopic observation revealed that more of the wild-type control mice had pulmonary edema at the end of the recovery period than Vegfd knockout mice; pulmonary edema was observed in 15 out of 21 wild-type control mice, but only in 4 out of 17 Vegfd knockout mice (p=0.0081, Fisher's exact test). FIG. 1 shows representative examples of the lungs of Vegfd knockout and wild-type control mice. These observations were supported by gravimetric analysis of extra-vascular lung water (EVLW), an indicator of the degree of pulmonary edema. The EVLW of Vegfd knockout mice was lower than that of wild-type control mice (0.116±0.003 g for Vegfd knockout mice, N=17; 0.129±0.004 g for wild-type control mice, N=20; P=0.008, t test), indicating that the lungs of wild-type control mice were more edematous than those of Vegfd knockout mice.

These findings demonstrate that endogenous VEGF-D in the mouse lung enhances the degree of pulmonary edema that occurs in response to hyperoxic exposure.

The findings described here suggest that in clinical settings where treatment with high oxygen concentrations is required (e.g. ALI/ARDS), inhibitors of VEGF-D or of its cognate receptors (VEGFR-3 or VEGFR-2), or of the closely related growth factor VEGF-C, may be therapeutically useful for treating pulmonary edema.

### Example 2

This study will assess VEGFR-3 signaling as a target for reducing pulmonary edema that develops in response to hyperoxia. The study will use a neutralizing monoclonal antibody (mAb) that binds specifically to mouse VEGFR-3 (for example, mF4-31C1; Pytowski et al., 2005, J Natl Cancer Inst, 97: 14-21) to reduce signaling via this receptor in the setting of hyperoxia. Alternatively, a neutralizing mAb that binds specifically to mouse VEGF-D may be used, either alone or in combination with a neutralizing mAb that binds specifically to mouse VEGF-C. In another alternative, a neutralizing mAb that binds specifically to mouse VEGF-C may be used. In yet more alternatives, a soluble VEGFR-3 construct (i.e. receptor trap) may be used to sequester both VEGF-D and VEGF-C, or a small molecule inhibitor of VEGFR-3 may be used.

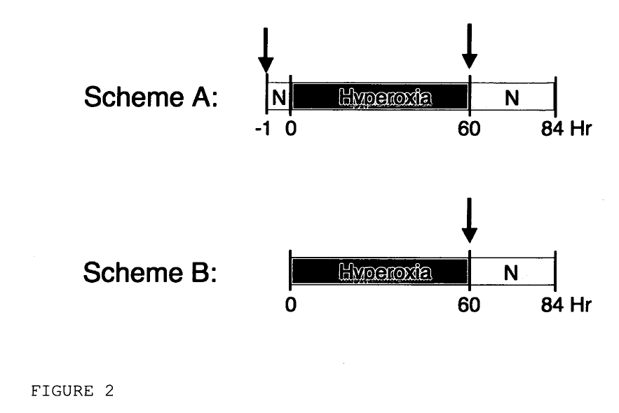

The neutralizing mAb that binds specifically to mouse VEGFR-3 (mF4-31C1) or an isotype-matched control mAb (or antiserum) will be injected into wild-type mice once, one hour before the beginning of the 60-hour hyperoxic exposure (approximately 95% O₂), and once thereafter, at the start of the 24-hour normoxic recovery period (FIG. 2, Scheme A), or will be injected only at the start of the recovery period (FIG. 2, Scheme B). Administration may be, for example, by intraperitoneal or intravenous injection. Intravenous injection (e.g. in the tail vein) may provide a more preferred mode of administration, resulting in a more rapid uptake of the mAb into the lungs. Inclusion of both Scheme A and B will allow monitoring of the effect of VEGFR-3 signaling on development of pulmonary edema and on its resolution. Mice will be sacrificed at the end of the 24-hour normoxic recovery period, and pulmonary edema will be detected by macroscopic observation initially, since the pulmonary edema will be indicated by swelling and discoloration of the lung tissue. The degree of pulmonary edema will then be determined by gravimetric analysis of the lungs to determine the extra-vascular lung water (EVLW), a well-established indicator of pulmonary edema.

Injection of the mF4-31C1 mAb according to Scheme A will lead to a reduction in the number of mice that develop pulmonary edema, as assessed by macroscopic observation at the end of the normoxic recovery period, in comparison to injection of the control mAb. The lungs of the mice injected with mF4-31C1 according to Scheme A will have a lower mean EVLW at the end of the normoxic recovery period than mice injected with control mAb. Mab mF4-31C1 will also reduce pulmonary edema in mice treated according to Scheme B, as determined by macroscopic observation and measurement of EVLW, although the degree of reduction of pulmonary edema will be less pronounced than for Scheme A. These findings will indicate that reducing VEGFR-3 signaling will restrict development of hyperoxia-induced pulmonary edema, and therefore reducing VEGFR-3 signaling will be useful for treating pulmonary edema.

### Example 3

A candidate compound may be screened for inhibitor activity using a cell proliferation bioassay that detects inhibition of interaction between a ligand and VEGFR-3. Therefore, such a bioassay can detect inhibition of signaling mediated by VEGFR-3. To this end, a bioassay was established in which Ba/F3 cells were stably transfected with a chimeric receptor containing the extracellular domain of human VEGFR-3 and the transmembrane and cytoplasmic domains of the mouse erythropoietin receptor (EpoR) (Achen et al. 2000 Eur J Biochem 267: 2505-2515). The chimeric receptor was made by introducing a BglII restriction enzyme site at the junction of the regions encoding the extracellular and transmembrane domains of the mouse EpoR cDNA using site-directed mutagenesis. Prior to this, a silent mutation was introduced into the EpoR cDNA in a region encoding the cytoplasmic domain of the EpoR to eliminate a naturally occurring BglII site. The fragment of the EpoR cDNA encoding the transmembrane and cytoplasmic domains, subcloned in plasmid pcDNA1/Amp (Invitrogen), was then ligated at the BglII site with a PCR product, consisting of DNA encoding the entire extracellular domain of human VEGFR-3, to generate a cDNA encoding a fusion protein consisting of the VEGFR-3 extracellular domain and the transmembrane and cytoplasmic domains of EpoR. The DNA fragment encoding the extracellular domain of human VEGFR-3 had been amplified by PCR using primers 5′-TAGAAAGCTTAATCTAGAGCCACCATGCAGCGGGGCG and 5′-TAGAGGATCCCTCCATGCTGCCCT and ligated as a HindIII-BamHI fragment into HindIII-BglII sites of the EpoR plasmid construct. The DNA encoding the chimeric receptor was subcloned into the expression vector pEF-BOS and cotransfected into the Ba/F3 cell line with pgk-Neo (a plasmid containing a neomycin resistance gene under the control of the promoter of the mouse phosphoglycerate kinase-1 gene), at a ratio of 20:1. Transfected cells were selected in G418, and a cell line expressing the VEGFR-3-EpoR chimeric receptor (Mᵣ≈150 000) was identified by immunoprecipitation and western blot analysis with antihuman VEGFR-3 polyclonal antibody (R & D Systems, Minneapolis, Minn., USA). Expression of the chimeric receptor was confirmed by flow cytometry using mAb 9D9 specific for the extracellular domain of human VEGFR-3. The cell line expressing the receptor was designated Ba/F3-VEGFR-3-EpoR. The Ba/F3 cell line is IL-3 dependent.

The chimeric molecule was used as members of the receptor-type tyrosine kinase family signal poorly in hematopoietic cells such as Ba/F3, whereas signaling from the EpoR cytoplasmic domain leads to cell survival and proliferation in the absence of IL-3. Ba/F3-VEGFR-3-EpoR can be rescued with a known VEGFR-3 ligand, for example VEGF-D, in the absence of IL-3. In contrast, the parental cell line, which does not express VEGFR-3, does not respond to the known VEGFR-3 ligand, indicating that the response of the Ba/F3-VEGFR-3-EpoR cell line to the known ligand is dependent on the chimeric receptor.

Thus, candidate compounds can be screened for the capacity to inhibit the activation of VEGFR-3 in such a cell proliferation bioassay using Ba/F3-VEGFR-3-EpoR cells. Compounds that reduce binding of the known ligand to the extracellular domains of the chimeric receptors or the subsequent cross-linking of the extracellular domains will cause cell death in the absence of IL-3, even in the presence of the known ligand.

Samples of a known ligand for VEGFR-3 will be incubated with a candidate compound for 1 h at 4° C. in NaCl/Pᵢ before dilution of the mixtures 1:10 with cell culture medium (e.g., Dulbecco's modified Eagle's medium containing 10% (v/v) fetal bovine serum, 50 mM L-glutamine, 50 μgmL⁻¹ gentamicin, 1 mgmL⁻¹ G418) deficient in IL-3. The resulting media will contain approximately 500 ngmL⁻¹ of known ligand for VEGFR-3 and varying concentrations of the candidate compound. Ba/F3-VEGFR-3-EpoR cells will be then incubated in the media for 48 h at 37° C. DNA synthesis will be then quantified by addition of 1 μCi of ³H-thymidine and further incubation for 4 h prior to harvesting, for example, using an automated cell harvester. Incorporated ³H-thymidine will be measured by β scintillation counting.

1. A method of treatment of pulmonary edema, comprising the step of administering to a subject in need thereof a therapeutically effective amount of an agent capable of inhibiting signaling mediated by VEGFR-3 in the subject.

2. The method of claim 1, wherein the agent is capable of inhibiting lymphangiogenesis in the subject.

3. The method of claim 1, wherein the agent is capable of inhibiting VEGF-D activity in the subject.

4. A method of treatment of pulmonary edema comprising the steps of:
identifying a subject at risk of developing pulmonary edema; and
administering to the subject a therapeutically effective amount of an agent capable of inhibiting signaling mediated by VEGFR-3 in the subject.

5. The method of claim 4,
wherein the agent is capable of inhibiting lymphangiogenesis in the subject.

6. The method of claim 4,
wherein the agent is capable of inhibiting VEGF-D activity in the subject.

7-8. (canceled)

9. The method according to claim 1, wherein the agent is administered to the subject as soon as is practicable after pulmonary edema initiates.

10. The method according to claim 1, wherein the agent is administered to the subject before pulmonary edema initiates.

11. The method according to claim 1, wherein the agent is selected from the group consisting of an inhibitor of VEGF-D, an inhibitor of VEGFR-3, an inhibitor of NRP-1, an inhibitor of NRP-2, an inhibitor of ANG-1, an inhibitor of TIE-2, an inhibitor of a proprotein convertase, and an inhibitor of integrin α9β1.

12. The method according to claim 11, wherein the inhibitor is selected from the group consisting of an antibody, a peptide, a peptidomimetic, a polypeptide, a nucleic acid molecule, and a small molecule.

13. The method according to claim 12, wherein the antibody comprises a monoclonal antibody, a polyclonal antibody, a chimeric antibody, a humanized antibody, a bispecific antibody, or fragment thereof, or a Fab, F(ab′)2, or scFv fragment.

14. The method, according to claim 12, wherein the antibody comprises a neutralizing monoclonal antibody.

15. The method according to claim 11, wherein the inhibitor comprises a soluble receptor.

16. The method according to claim 14, wherein the antibody comprises a neutralizing monoclonal anti-VEGF-D antibody.

17. The method according to claim 16, wherein the antibody is 4A5, or is a humanized antibody derived from antibody 4A5.

18. The method according to claim 14, wherein the antibody comprises a neutralizing monoclonal anti-VEGFR-3 antibody.

19. The method according to claim 18, wherein the inhibitor comprises a neutralizing monoclonal anti-VEGFR-3 antibody, wherein the antibody is hF4-3C5.

20. The method according to claim 1 wherein the pulmonary edema is cardiogenic.

21. The method according to claim 1 wherein the pulmonary edema is non-cardiogenic.

22. The method according to claim 1 wherein the pulmonary edema is associated with arrhythmia; atherosclerosis; arteriosclerosis; arteriovenous malformation; cardiomyopathy; cardiovascular disease; congestive heart failure; coronary artery disease; hypervolemia; heart attach; left ventricular failure; heart disease; hypertensive crisis; pulmonary hypertension; pericardial effusion; pericardial tamponade; or valvular heart disease.

23. The method according to claim 1 wherein the pulmonary edema is associated with acute lung injury; acute respiratory distress syndrome; sepsis; pneumonia; pancreatitis; trauma; aspiration; burns; inhalation of toxic or noxious gas or fumes; air embolism; amniotic fluid embolism; fat embolism; massive or multiple blood transfusions; eclampsia; poisoning; radiation; asthma; bronchopulmonary dysplasia; chronic obstructive pulmonary disease; high altitude pulmonary edema; hyperoxic acute lung injury; hypoventilation; some medications; narcotic (opioid) overdose; re-expansion pulmonary edema; reperfusion; or upper airway obstruction.

24. The method according to claim 1 wherein the pulmonary edema is associated with lung transplantation.

25. The method according to claim 1 wherein the subject is human.
